(19) United States
(12) Patent Application Publication
(10) Pub. No.: US 2007/0282285 A1
(43) Pub. Date: Dec. 6, 2007

(54) Phantom for collecting animal semen

(76) Inventor: Jean-Francois Yvoz, Steenvoorde (FR)

Correspondence Address: KNOBBE MARTENS OLSON & BEAR LLP, 2040 MAIN STREET, FOURTEENTH FLOOR, IRVINE, CA, 92614, US

(21) Appl. No.: 11/492,702

(22) Filed: Jul. 24, 2006

(30) Foreign Application Priority Data
Jun. 1, 2006 (EP) 06370020.7

(51) Int. Cl. A61F 5/44

(52) U.S. Cl. 604/349

(57) ABSTRACT

The phantom (1) for animals, in particular porcine animals, comprises a frame (2) to which the animal semen collection means (4) are attached. These collection means (4) are mobile between at least one animal semen collection position and a retracted position. The phantom (1) comprises means (7) for detecting the presence of an animal in position on the frame (2), and actuation means (6), which are designed to move the collection means between the collection position and the retracted position, and which are controlled by the detection means (7).

This invention relates to the field of animal semen collection. It relates to a new phantom used to collect the semen of an animal. It is primarily, but not exclusively, applied to the collection of boar semen.

### PRIOR ART

To collect the semen of an animal, such as, for example, a boar, a cow, a horse, etc., it is presently commonplace to use a collection phantom, which consists of a rigid frame that is placed on the ground, simulating the female, and on which the animal assumes its position with the rear portion of its body in contact with the frame, and the hind legs of the animal usually remaining on the ground. Animal semen collection means are also attached to this phantom frame, which means include, in particular, an artificial vagina that makes it possible to stimulate the animal's penis and cause ejaculation.

Examples of phantom structures for collecting animal semen are described, in particular, in American patents U.S. Pat. No. 4,744,352, U.S. Pat. No. 4,620,531, U.S. Pat. No. 5,685,871 and U.S. Pat. No. 6,090,088, and in international patent application WO 01/54491.

There are currently a wide variety of artificial vaginas with substantially complex structures, which can be mounted on the frame of a phantom. These artificial vaginas are generally adapted to the animal species from which the semen is to be collected.

For example, European patent application EP 0 699 422 proposes a single-use artificial vagina having a very simple structure, consisting of a tubular sheath made of non-toxic polyurethane, polyethylene or silicon foam. This artificial vagina having a very simple structure enables the animal semen to be collected manually, as the penis of the animal must be manually stimulated by the operator by means of pressure exerted using the tubular foam sheath.

More sophisticated artificial vaginas allowing for automated stimulation of the penis, in particular by means of a variable pressure chamber supplied with a fluid such as water or air, are also known.

For example, international patent application WO 02/13723 discloses an artificial vagina implementing a sealed air chamber and allowing for localised stimulation of the animal's penis by means of pulsations.

Artificial vaginas with automatic stimulation are advantageous because they facilitate the collection operation for the operator. They are therefore more suitable for implementation in a collection phantom.

To perform a collection operation, the operator in charge of the collection helps the animal to mount the frame of the phantom, then, once the animal is in position, inserts the animal's penis into the artificial vagina and begins the animal semen collection operation.

The animal has a tendency to dismount from the phantom alone, in particular once the ejaculation phase has been completed. If there is no human intervention when the animal dismounts from the phantom, there is an increased risk that the animal will damage the collection means, which is detrimental when the collection means implement an artificial vagina that is not for a single use, and in particular when the artificial vagina has a sophisticated structure allowing for automated stimulation of the penis. To avoid these risks of damage, the operator is currently required to monitor the animal during the entire collection operation. This requirement of continuous presence of the operator near the animal makes the task tiresome with porcine animals, in which the collection operation takes a long time (generally between 5 and 10 minutes). In addition, this requirement of continuous presence of the operator near the animal limits the number of animals capable of being simultaneously collected by a single operator, and therefore limits the collection rates and the productivity of an operator.

### OBJECTS OF THE INVENTION

This invention is intended to provide a new phantom for animal semen collection that enables the risk of accidental damage of the collection means by the animal to be reduced, without requiring the presence of the collection operator.

### SUMMARY OF THE INVENTION

The aforementioned objective is achieved by the collection phantom of claim 1. This phantom for animals, in particular porcine animals, comprises a frame to which the animal semen collection means are attached; the collection means are mobile between at least one animal semen collection position and a retracted position, and the phantom comprises means for detecting the presence of an animal in position on the frame, and actuation means, which are designed to move the collection means between the collection position and the retracted position, and which are controlled by the detection means.

Once the animal semen collection operation has been completed, the animal dismounts alone from the frame of the phantom. In this case, the detection means detect the absence of the animal, and automatically control the actuation means so as to bring the collection means to their retracted position. This automatic retraction of the collection means advantageously makes it possible to remove the animal semen collection means and, as the case may be, the semen collected, without human intervention.

Thus, the risks of accidental damage of these collection means by the animal, and, as the case may be, the loss of the collected semen, are avoided.

Preferably, when the animal is in position on the frame of the phantom, the detection means detect the presence of the animal, and automatically control the actuation means so as to bring the collection means to their collection position.

More specifically, the phantom of the invention can also comprise the additional and optional features mentioned in the appended claims, which are dependent on the main claim 1.

### BRIEF DESCRIPTION OF THE FIGURES

Other features and advantages of the invention will become more clear in the following detailed description of a preferred alternative of a phantom of the invention, which description is given by way of a non-limiting and non-exhaustive example of the invention, in reference to the appended drawings, wherein:

FIG. 1 shows a perspective view of a preferred alternative of a phantom of the invention, with the collection means in their collection position,

FIG. 2 shows the phantom of FIG. 1, with a transparent view of the (phantom) frame, so as to show the elements attached under this frame by transparency,

FIG. 3 shows the phantom of FIG. 1, when the collection means are in their retracted position,

FIG. 4 shows the phantom of FIG. 1, without its frame,

FIG. 5 shows the phantom of FIG. 3, when the frame of this phantom is tilted with respect to its ground support, so as to make the underpart of the frame more easily accessible,

FIG. 6 is a transverse cross-section view of the guide rail allowing for a guided translation movement of the collection means, and means providing the connection between this guide rail and the collection means,

FIG. 7 is a simplified diagram of the detection means enabling the actuation means to be controlled for automatic movement of the collection means.

### DETAILED DESCRIPTION

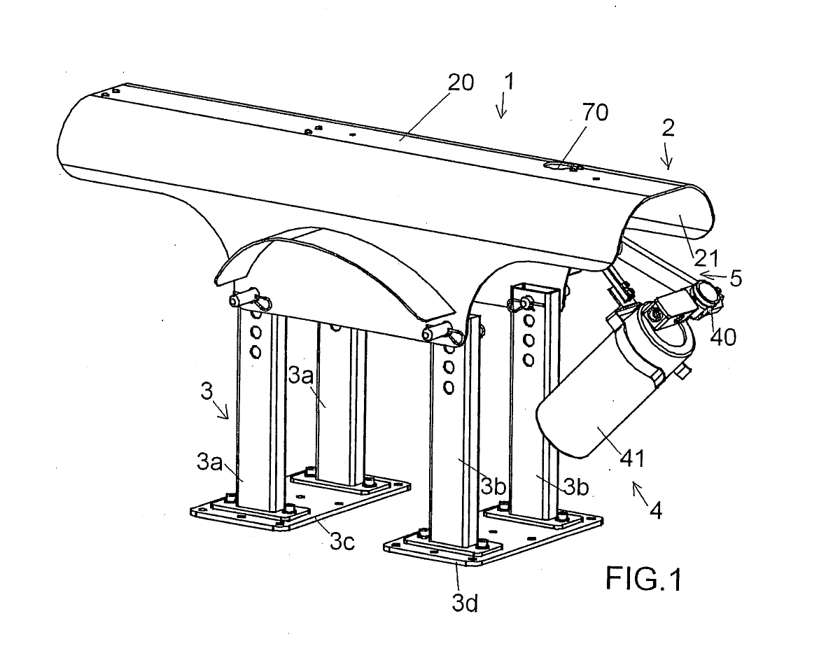

FIGS. 1 to 5 show an animal semen collection phantom 1 that corresponds to a preferred alternative of the invention.

This phantom 1 comprises a rigid frame 2 commonly referred to as a “phantom”, which is supported in a stable manner on the ground by support means 3. This frame 2 comprises a wall 20 that usually enables the female to be simulated while supporting the (male) animal during the animal semen collection operation. In the specific example shown, this wall 20 has a U-shaped profile. In the context of the invention, the structure, shape and material(s) constituting this frame 2 are unimportant. This frame 2 can in particular be made up of a single part or an assembly of a plurality of parts.

In the specific example of the appended figures, the support means 3 comprise four vertical legs 3a, 3b. To increase the ground stability of the phantom 1, the two front legs 3a are preferably attached to a base 3c, and the two rear legs 3b are attached to a base 3d. The invention is not, however, limited to this specific structure, as the support means 3 can be replaced by any equivalent means, having a different structure, enabling the frame 2 to be supported on the ground.

The phantom 1 also comprises animal semen collection means 4, which are attached tot he frame 2, and more specifically, which are attached to the lower surface 21 of the wall 20 of the frame 2. These collection means 4 comprise an artificial collection vagina 40 that makes it possible to simulate the vagina of the female, associated with a container 41 for collecting animal semen flowing by gravity from the artificial vagina 40.

Preferably, and in a manner that is non-limiting to the invention, the artificial vagina 40 allows for automatic stimulation of the animal's penis, and, for example, corresponds to the artificial vagina described in the international patent application WO 02/13723.

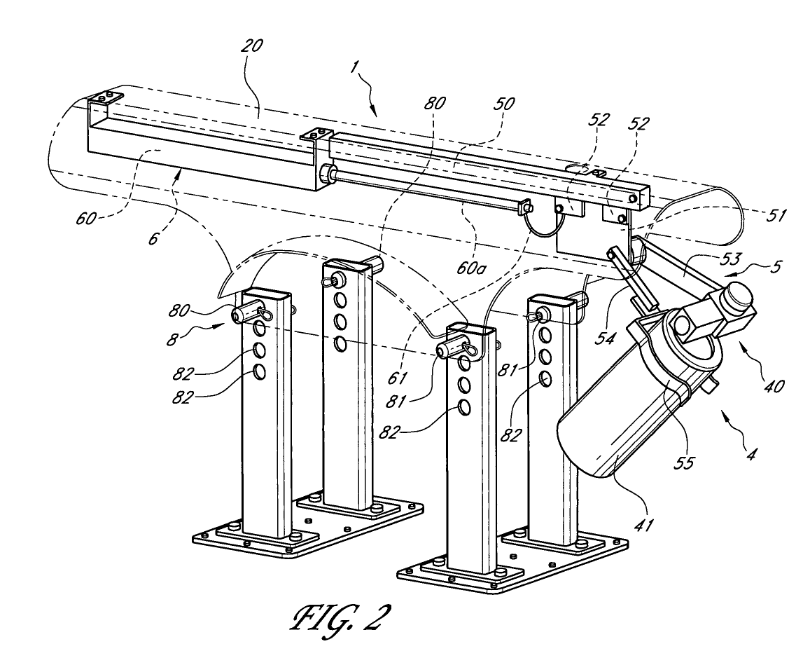

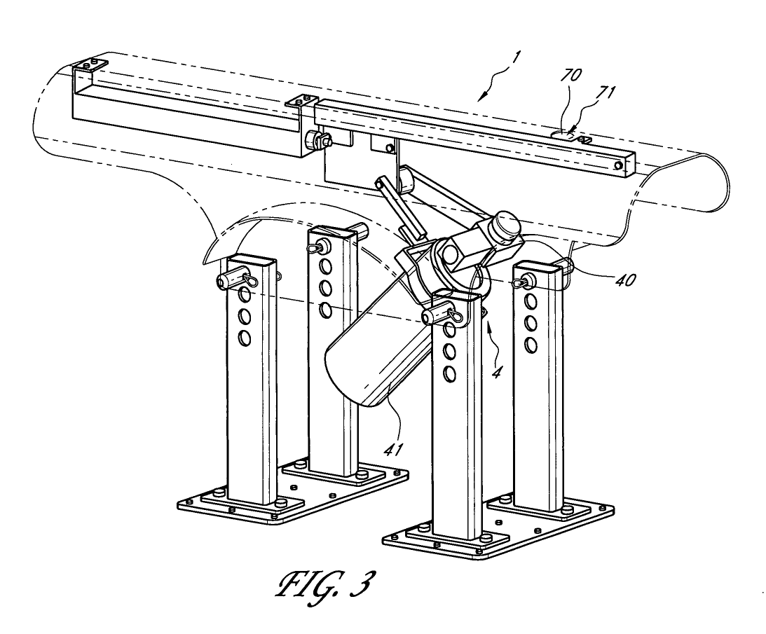

According to a feature of the invention, the collection means 4 can move with respect to the frame 2, between two positions: a collection position shown in FIGS. 1 and 2, and a so-called “retracted” position, shown in FIG. 3.

A specific example of mechanical connection means 5 between the frame 2 and the collection means 4 will now be described in detail, with the understanding that this example does not limit the invention, and that a person skilled in the art can replace the particular mechanical connection means 5 of the appended figures with any equivalent connection means performing the same function, i.e. enabling the collection means 40 to move between two different positions.

In reference to FIGS. 1 to 5, the connection means 5 comprise a rail 50 that forms a support and guide slide, and which is attached under the frame 2, and a plate 51, which is supported by the rail 50, by means of two connecting parts 52, and which can be moved in translation along the rail 50. Two arms 53 and 54 are attached to this mobile plate 51. The artificial vagina 40 is attached to the end of the distal arm 53 of the plate 51. The collection container 41 is attached to the end of the distal arm 54 of the plate 51, preferably so as to be easily removable, by means of a gripper 55, for example. The collection means 4 (artificial vagina 40/container 41) held by the mobile plate 51 are thus mobile and guided in translation along the rail 50.

The movement of the collection means 4 along the rail 50 is controlled by actuation means 6. In the specific example shown, these actuation means 6 comprise a linear actuator 60 with two positions (for example, a pneumatic actuator), which is attached under the frame 2. The mobile rod 60a of the actuator 60 is secured to the mobile plate 51 by means of a flexible link 61.

The movement of the collection means 4 to the retracted position is achieved by pulling on the mobile plate 51, by means of the mobile rod 60a of the actuator 60 and the flexible link 61, until a position (FIG. 3) is reached in which the collection means 4 are positioned entirely under the frame 2.

The movement of the collection means 4 to the collection position is achieved by pushing the mobile plate 51 by means of the rod 60a of the actuator 60, to a position (FIGS. 1 and 2) in which the collection means 4 at least partially go beyond the frame 2. In the specific example shown, in the collection position, the artificial vagina 40 is no longer located under the frame 2, and is therefore easily accessible.

It should be noted that in this collection position, owing to the implementation of the flexible link 61, the collection means 4 can move freely in translation along the rail 50, and over a short path, in an out-and-back movement. This mobility of the collection means 4 in the collection position advantageously makes it possible, in the animal semen collection operation, to give the artificial vagina 40 a to-and-fro movement under the action of the animal's penis, which facilitates the animal's ejaculation. In addition, it is not necessary to apply mechanical stress on the mobile rod of the actuator 60, during the collection operation.

In another, less developed, alternative of the invention, the collection means 4 can be rigidly attached to the actuation means 6.

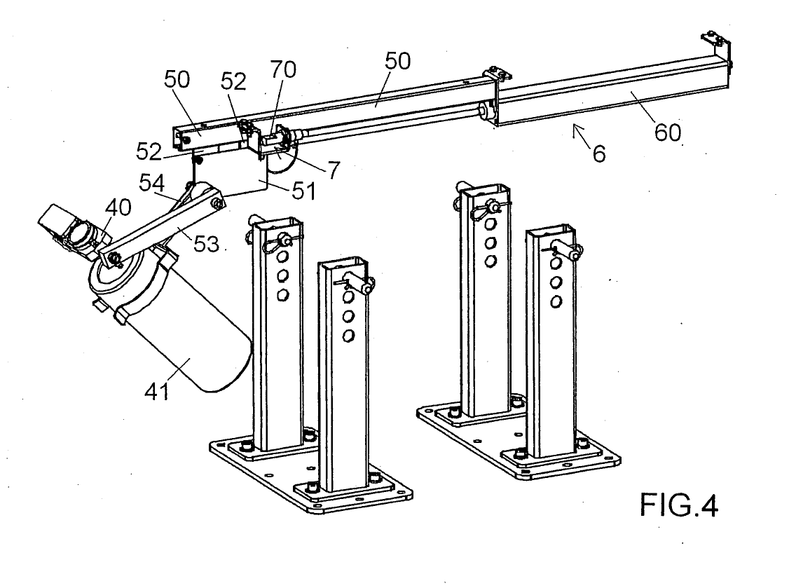

In reference to FIGS. 3 and 4, the phantom 1 is equipped with means 7 for detecting the presence of an animal in position on the frame 2.

In the particular example of the appended figures, these detection means 7 comprise a photoelectric cell-type contactless sensor 70, which is attached under the frame 2, opposite a through-hole 71 formed in the frame 2. In reference to FIG. 7, this sensor 70 generates an electrical detection signal 70a, which is processed by electronic means 72. These electronic means 72 are designed to control the actuator 60 by means of a control signal 72a, according to the state of the sensor 70.

In operation, the operator in charge of the collection mounts the animal onto the frame 2 of the phantom, with the collection means 4 being housed under the frame 2 (retracted position).

When the animal is in position on the frame 2 of the phantom 1, the hole 71 is closed off, and the sensor 70, which is no longer illuminated, automatically detects the presence of the animal (modification of the detection signal 70a). The resulting modification of the signal 70a is detected by the electronic means 72, which electronic means 72 are designed to automatically control the actuator 60 so as to automatically move the collection means 4 into their collection position (removal of the rod 60a from the actuator).

Once the collection means 4 are in their collection position as shown in FIGS. 1 and 2, the operator inserts the animal's penis into the artificial vagina 40, and the animal semen collection operation can be performed as usual.

When the animal has ejaculated, he instinctively dismounts from the frame 2 of the phantom 1. The hole 71 is then no longer closed off and the sensor 70 is illuminated, which results in a modification of the detection signal 70a. This modification of the signal 70a is detected by the electronic means 72, which electronic means 72 are designed to automatically control the actuator 60 so as to automatically move the collection means 4 into their retracted position (retraction of the rod 60a of the actuator 60 into the actuator body).

This automatic movement of the collection means 4 into the retracted position under the frame 2 advantageously makes it possible to protect the collection means 4 from any accidental damage by the animal. The collected semen is also protected. This advantageously means that the operator is no longer required to monitor the animal during the entire collection operation, which improves the productivity of the collection operations. The collection operation advantageously also becomes less tiresome and more reliable for the operator.

The electronic means 72 can be implemented in various ways. In a non-limiting and non-exhaustive example of the invention, they can be produced in the form of an electronic card comprising a processor (microprocessor or a microcontroller) designed to run a program (automatic control operation) that is stored in a memory, and that, when run by the processor, makes it possible to implement the aforementioned steps of the operation; the electronic means 72 can also be implemented by means of an industrial programmable automaton (IPA).

In the context of the invention, the detection means 7 can be produced with any type of sensor or set of sensors. The invention is not limited to the implementation of one or more contactless sensor(s), but can also be produced with one or more contact sensor(s). In addition, it is possible to implement, as detection means, one or more sensors enabling the weight supported by the frame 2 of the phantom 1 to be detected.

In another alternative of the invention, the detection means 7 and the actuation means 6 for the automatic movement of the collection means 4 between the collection position and the retracted position can be solely mechanical.

In another alternative of the invention, only the retraction movement of the collection means can be automated, with the movement of the collection means from the retracted position to the collection position being performed manually by the operator.

The retraction movement of the collection means 4 is not necessarily a translation movement as shown in the alternative of the appended figures. It can involve any other type of movement or combination of movements (translation, rotation, nutation, etc.).

In the retracted position, the collection means are preferably, but not necessarily, housed entirely under the frame 2. In another alternative of the invention, the collection means 4 can be brought to a retracted position, which is far enough from the collection position so that they will not be easily in contact with the animal when the latter dismounts from the frame of the phantom, but nevertheless without the collection means 4 being protected by the frame in this retracted position.

In the alternative described in reference to the appended figures, the artificial vagina 40 and the container constitute the collection means 4 and are mobile between the collection position and the retracted position. In another alternative, only the artificial vagina can be retractable.

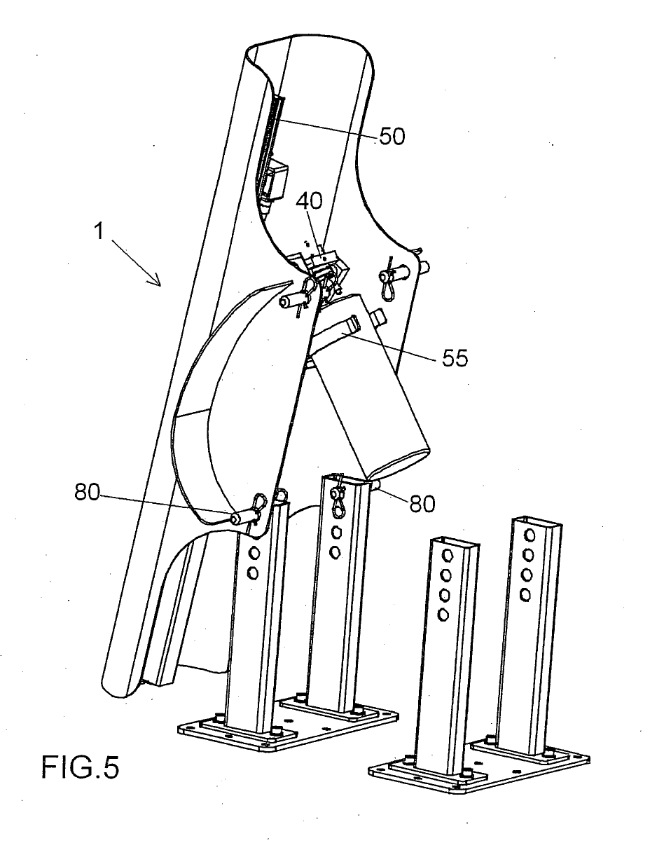

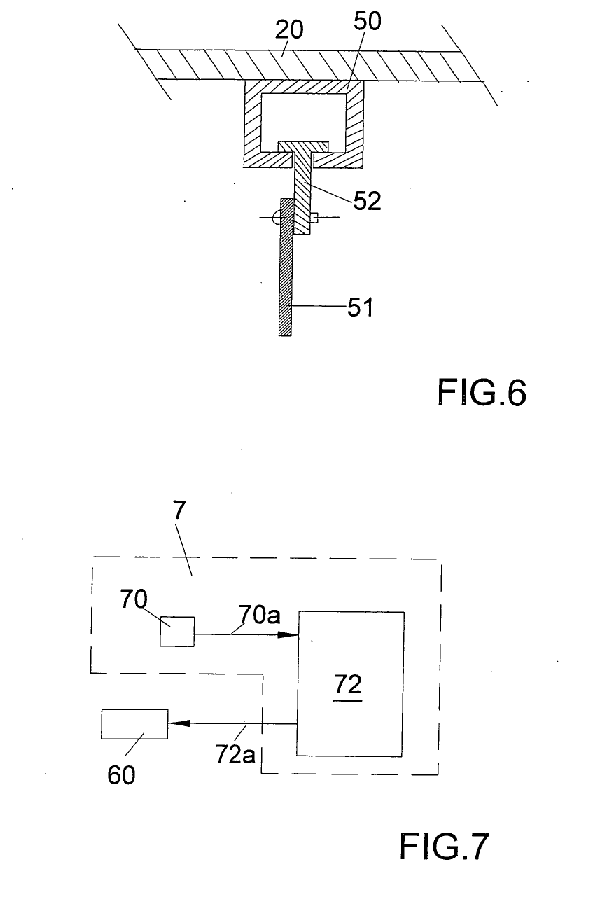

The means 8 for connection between the frame 2 and the support means 3 preferably, but not necessarily, comprise means 80 enabling the frame 2 to pivot with respect to the support means 3, and means 81 enabling the pivoting of the frame 2 to be blocked or unblocked. In the particular example shown in the appended figures, the pivoting means 80 consist of two rotation pins passing through the wall 20 of the frame 2 and the front legs 3a, respectively; the blocking/unblocking means 81 consist of two blocking pins that pass through the wall 20 of the frame 2 and the rear legs 3b, respectively, and that are removable. When these blocking pins 81 are removed from the frame 2, said frame 2 can advantageously pivot with respect to the front legs 3a (see FIG. 5), so that the underpart of this frame 2 is easily accessible and all of the elements secured to the frame 2, in particular the sensor 70, the actuation means 6, the connection means 5, and the collection means 4, are more easily accessible, and can, for example, be more easily cleaned.

The frame 2 is preferably, but not necessarily, vertically adjustable with respect to the support means 3, so as to match the phantom to the animal's size. In the particular example shown, this vertical adjustment is achieved by providing, in each leg 3a, 3b, a series of adjustment through-holes 82, allowing for removable positioning of the pins 80 and 81.

The phantom of the invention is particularly suitable for collecting the semen of animals characterised by a relatively long ejaculation phase, and is particularly suitable for collecting the semen of boars.

What is claimed is:

1. An apparatus for collecting semen from an animal, comprising:
a frame;
a semen collector attached to the frame and moveable between at least one semen collection position and a retracted position;
a detector for detecting the presence of an animal when in position on the frame; and
an actuator adapted to move the collector between the collection position and the retracted position and controlled by the detector.

2. An apparatus according to claim 1, wherein the detector and the actuator are adapted to automatically move the collector into the retracted position when the presence of an animal in position on the frame is no longer detected.

3. An apparatus according to claim 1, wherein the detector and the actuator are adapted to automatically move the collector into the collection position when the presence of an animal in position on the frame is detected.

4. An apparatus according to claim 1, wherein the collector comprises an artificial vagina.

5. An apparatus according to claim 4, wherein the artificial vagina is adapted to provide automatic stimulation of the animal's penis.

6. An apparatus according to claim 4, wherein the collector comprises a receptacle associated with the artificial vagina.

7. An apparatus according to claim 1, wherein the frame comprises a support structure enabling an animal to be supported, the support structure having a lower surface to which the collector is attached.

8. An apparatus according to claim 1, wherein in the retracted position, the collector is housed entirely under the frame.

9. An apparatus according to claim 1, wherein the collector is moveable in translation with respect to the frame.

10. An apparatus according to claim 1, wherein the actuator is connected to the collector by a flexible link.

11. An apparatus according to claim 1, wherein the detector comprises a contactless sensor.

12. An apparatus according to claim 11, wherein the frame comprises a through-hole in which the sensor is attached to the frame.

13. An apparatus according to claim 1, further comprising a support structure attached to the frame and wherein the frame is pivotally mounted to the support structure.

14. An apparatus according to claim 13, further comprising a release mechanism to allow the frame to pivot.

15. An apparatus according to claim 13, wherein the frame is vertically adjustable with respect to the support structure.
